(19) United States
(12) Patent Application Publication
(10) Pub. No.: US 2012/0065255 A1
(43) Pub. Date: Mar. 15, 2012

(54) CABAZITAXEL FORMULATIONS AND METHODS OF PREPARING THEREOF

(76) Inventor: Nagesh Palepu, Southampton, PA (US)

(21) Appl. No.: 13/207,334

(22) Filed: Aug. 10, 2011

Related U.S. Application Data
(63) Continuation-in-part of application No. 12/721,564, filed on Mar. 11, 2010.
Application No. 12/721,564 is a continuation-in-part of application No. 12/589,145, filed on Oct. 19, 2009.

(51) Int. Cl. A61K 31/337; A61P 35/00

(52) U.S. Cl. 514/449

(57) ABSTRACT

Pharmaceutical formulations comprising cabazitaxel, solubilizer, tocopherol polyethylene glycol succinate (TPGS), one or more hydrotropes, optionally one or more agents having a pKₐ of about 3 to about 6, and optionally one or more antioxidizing agents, wherein the formulations are substantially free of polysorbates and polyethoxylated castor oil. The solubilizer may comprise glycofurol or ethanol. Pharmaceutical formulations may alternatively comprise cabazitaxel, solubilizer, optionally one or more agents having a pKₐ of about 3 to about 6, and optionally one or more antioxidizing agents, wherein the formulations are substantially free of polysorbates and polyethoxylated castor oil. These formulations may be combined with a diluent, which comprises TPGS and one or more hydrotropes. Methods of administering the cabazitaxel formulations include combining the formulations with an infusion solution.

### CROSS REFERENCE TO RELATED APPLICATIONS

This application is a continuation-in-part of U.S. patent application Ser. No. 12/721,564 (“'564 application”), filed on Mar. 11, 2010, which is a continuation-in-part of U.S. patent application Ser. No. 12/589,145 (“145 application”), filed on Oct. 19, 2009. Both the '564 application and the '145 application are incorporated by reference.

### FIELD OF THE INVENTION

The present invention relates to pharmaceutical formulations comprising cabazitaxel, and a solubilizer, wherein the formulation is substantially free of polysorbates and polyethoxylated castor oil. The present invention also relates to pharmaceutical formulations comprising cabazitaxel, a solubilizer, and a diluent. The diluent may comprise one or more hydrotropes. In addition, the present invention relates to pharmaceutical formulations comprising cabazitaxel, a solubilizer, a diluent, and an infusion solution in order to administer the formulation to patients. The present invention further relates to formulations comprising other taxane or hard to solubilize molecules. In addition, the present invention relates to methods for administering the formulations to patients in need thereof and for preparing the formulations.

### BACKGROUND OF THE INVENTION

Antineoplastic agents inhibit and combat the development of neoplasms, which are abnormal masses of tissue resulting from irregular proliferation of cells. Such antineoplastic agents include cabazitaxel, a taxane compounds derived from the renewable needle biomass of yew plants. Cabazitaxel binds to free tubulin and promotes the assembly of microtubules, which reduces the availability of tubulin for, and thereby prevents, cell division. Simultaneously, cabazitaxel inhibits microtubule disassembly, causing apoptosis. See JEVTANA® Prescribing Information.

Cabazitaxel is marketed as JEVTANA®, which is indicated in combination with prednisone for the treatment of patients with hormone-refractory metastatic prostate cancer previously treated with docetaxel. JEVTANA is supplied as a kit consisting of (a) a JEVTANA injection, which contains 60 mg cabazitaxel in 1.5 mL polysorbate 80; and (b) a diluent, containing approximately 5.7 mL 13% (w/w) ethanol. Prior to administration, the JEVTANA injection must first be mixed with the diluent, which dilutes the amount of cabazitaxel to 10 mg/mL, and then further diluted into a 250 mL PVC-free container of either 0.9% sodium chloride solution or 5% dextrose solution for infusion. The concentration of cabazitaxel in the resulting final infusion solution should be between 0.10 mg/mL and 0.26 mg/mL. See JEVTANA Prescribing Information.

Other taxane compounds include docetaxel, which is marketed as TAXOTERE® and is FDA-approved for breast cancer, non-small cell lung cancer, hormone refractory prostate cancer, gastric adenocarcinoma, and squamous cell carcinoma of head and neck cancer. TAXOTERE is available as a single-vial injection concentrate that is ready to add to an infusion solution. See TAXOTERE Prescribing Information.

The presence of polysorbate 80 in JEVTANA, as well as TAXOTERE, can result in serious side effects. Such reactions characterized by generalized rash/erythema, hypotension and/or bronchospasm, or very rarely fatal anaphylaxis, have been reported in patients. Hypersensitivity reactions may require immediate discontinuation of the taxane infusion and administration of appropriate therapy.

In order to reduce the side effects induced by polysorbate 80, patients may be treated with dexamethasone prior to each dose of JEVTANA. Dexamethasone is a steroid that suppresses the immune response in patients, which can be especially detrimental in cancer patients under chemotherapy, whose immunity may already be compromised due to the destruction of healthy cells by the chemotherapeutic treatment. As a result, these patients can be susceptible to bacterial and fungal infections. Further, despite receiving the dexamethasone premedication, patients can report hypersensitivity side effects from the taxane compound treatment.

Due to these side effects, patients may stop taxane compound therapy, skip a dose, or continue further therapy at a reduced dose. Similarly, other solubilizing agents such as CREMOPHOR EL®, which is a polyethoxylated castor oil used in connection with the marketed paclitaxel product TAXOL®, induce similar allergic reactions requiring premedication with a steroid.

Therefore, new formulations of cabazitaxel or other taxane compounds are needed to avoid these side effects, premedication requirements, and patient noncompliance issues associated with the currently marketed formulation.

### SUMMARY OF THE INVENTION

Applicant has developed stable formulations of cabazitaxel that are at least substantially free of polysorbates and polyethoxylated castor oil. These cabazitaxel formulations may be (a) single-vial injection concentrates, which are sterile liquids in a single vial ready to dilute with an infusion solution; (b) dual-vial injection concentrates, which require mixing with a diluent before it can be further diluted with an infusion solution; (c) diluted injection concentrate, which are dual-vial injection concentrates mixed with the special diluent; and (d) final dilutions for infusion, which are the single-vial injection concentrates or the diluted injection concentrates, combined with an infusion solution and ready to be administered. Applicant has also developed a kit comprising a dual-vial injection concentrate and a diluent for preparing the diluted injection concentrate, methods for administering cabazitaxel to patients in need thereof, and methods for preparing the cabazitaxel formulations.

Therefore, an aspect of the invention may relate to a sterile pharmaceutical formulation for use in treatment of a patient in need thereof, such as a single-vial injection concentrate, comprising (a) cabazitaxel, or a pharmaceutically acceptable salt thereof; (b) solubilizer, wherein the solubilizer is selected from glycofurol and ethanol; (c) tocopherol polyethylene glycol succinate (TPGS); (d) one or more hydrotropes; (e) optionally one or more agents having a pKₐ of about 3 to about 6; and (f) optionally one or more antioxidizing agent; wherein the formulation is substantially free of polysorbates and polyethoxylated castor oil.

Another aspect of the invention may relate to a method of preparing a sterile cabazitaxel formulation, such as a single-vial injection concentrate, substantially free of polysorbates and polyethoxylated castor oil, such that the method comprises combining together: (a) cabazitaxel, or a pharmaceutically acceptable salt thereof; (b) solubilizer, wherein the solubilizer is selected from glycofurol and ethanol; (c) tocopherol polyethylene glycol succinate (TPGS); (d) one or more hydrotropes; (e) optionally one or more agents having a pKₐ of about 3 to about 6; and (f) optionally one or more antioxidizing agent.

In some embodiments, cabazitaxel is in an amount of about 8 to about 12 mg/mL, or about 10 mg/mL.

In certain embodiments, the solubilizer is about 10 to about 35% of the total volume of the formulation. In some embodiments, the solubilizer is glycofurol.

In further embodiments, the TPGS is in an amount of about 0.15 to about 0.35 g/mL.

In certain embodiments, the one or more hydrotropes is at least polyethylene glycol (PEG), such as PEG 200, 300, 400, or 800. In some embodiments, PEG is in an amount to q.s. the formulation to its total volume.

In certain embodiments, the pharmaceutical formulation comprises one or more agents having a pKₐ of about 3 to about 6. In some embodiments, the one or more agents having a pKₐ of about 3 to about 6 is in an amount of about 0.3 to about 0.5 mg/mL. In some embodiments, the one or more agents having a pKa of about 3 to about 6 is at least an acid. In some embodiments, the acid is citric acid. In certain embodiments, the acid is a carboxylic acid, such as an aliphatic acid, e.g., acetic acid, or such as an aromatic acid, e.g., succinic acid. In some embodiments, the acid is a hydroxy carboxylic acid, such as an aliphatic acid, e.g., lactic acid, or such as an aromatic acid, e.g., salicylic acid.

In certain embodiments, the pharmaceutical formulation comprises one or more antioxidizing agents. In some embodiments, the one or more antioxidizing agents is at least α-lipoic acid. In further embodiments, the α-lipoic acid is in an amount of about 0.2 to about 1 mg/mL.

In certain embodiments, the pharmaceutical formulation may further comprise water for injection. In some embodiments, the formulation is substantially free of precipitates.

Yet another aspect of the invention relates to a sterile pharmaceutical formulation for use in treatment of a patient in need thereof, such as a dual-vial injection concentrate, comprising: (a) cabazitaxel, or a pharmaceutically acceptable salt thereof; (b) solubilizer, wherein the solubilizer is selected from glycofurol and ethanol; (c) optionally one or more agents having a pKa of about 3 to about 6; and (d) optionally one or more antioxidizing agents. In some embodiments, the solubilizer is glycofurol. In certain embodiments, the pharmaceutical formulation comprises one or more agents having a pKₐ of about 3 to about 6. In certain embodiments, the one or more agents having a pKₐ of about 3 to about 6 is citric acid. In further embodiments, the one or more agents having a pKₐ of about 3 to about 6 is a carboxylic acid or a hydroxy carboxylic acid. In some embodiments, the pharmaceutical formulation further comprises a diluent.

In certain embodiments, the diluent comprises 13% ethanol, TPGS, or a combination of thereof. In some embodiments, the TPGS is TPGS 1000. In further embodiments, the TPGS is in an amount of at least about 200 mg/ml. In yet further embodiments, the diluent also comprises one or more hydrotropes. In some embodiments, the one or more hydrotropes is at least PEG. In certain embodiments, the PEG is PEG 300, 400 or 800.

Another aspect of the present invention relates to a kit comprising a sterile pharmaceutical formulation, such as a dual-vial injection concentrate, and a diluent. In some embodiments, the pharmaceutical formulation comprises: (a) cabazitaxel, or a pharmaceutically acceptable salt thereof; (b) solubilizer, wherein the solubilizer is selected from glycofurol and ethanol; (c) optionally one or more agents having a pKa of about 3 to about 6; and (d) optionally one or more antioxidizing agents, as described above. In some embodiments, the pharmaceutical formulation comprises one or more agents having a pKa of about 3 to about 6, and/or one or more antioxidizing agents. In certain embodiments, the diluent comprises 13% ethanol, TPGS, or a combination thereof, and one or more hydrotropes, as described above.

A further aspect of the present invention relates to administration of a pharmaceutical formulation of the invention to a patient in need thereof.

These and other embodiments are disclosed or are obvious from and encompassed by, the following Detailed Description.

### DETAILED DESCRIPTION

The present invention is directed to sterile cabazitaxel formulations of single-vial injection concentrates, dual-vial injection concentrates, diluted injection concentrates, and final dilutions for infusion; to kits comprising a cabazitaxel dual-vial injection concentrate and a diluent for preparing the diluted injection concentrate; and methods for administering cabazitaxel to patients in need thereof and for preparing the cabazitaxel formulations.

Cabazitaxel is classified as a taxane, a class of compounds that can eradicate cancer cells. This class of compounds is virtually insoluble in water; therefore, in order to formulate taxanes for intravenous (IV) administration to patients, the formulations must include an excipient that will solubilize the taxane in solution. Further, since taxanes are cytotoxic, these solutions must be diluted before administrating to patients, but the dilution must not cause the taxane to precipitate, especially after dilution with an infusion solution for IV administration, or during administration of the taxane into the patient's bloodstream. Moreover, the selected excipients must allow the taxane to be administered by the IV route to a patient safely and effectively, with minimal side effects. To this end, Applicant has developed formulations that will solubilize cabazitaxel, prevent its precipitation upon dilution, and avoid the use of polysorbates and polyethoxylated castor oil, which as described above most often induce side effects in patients and require premedication.

### DEFINITIONS

As used herein, “cabazitaxel” refers to a drug substance having the chemical name of (2α,5β,7β,10β,13α)-4-acetoxy-13-({(2R,3S)-3-[(tertbutoxycarbonyl)amino]-2-hydroxy-3-phenylpropanoyl}oxy)-1-hydroxy-7,10-dimethoxy-9-oxo-5,20-epoxytax-11-en-2-yl benzoate-propan-2-one(1:1). Cabazitaxel has the following structural formula:

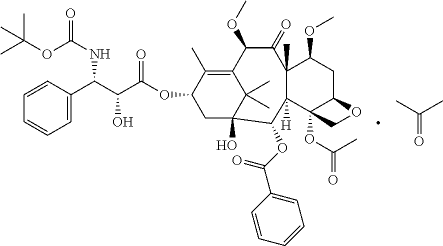

Cabazitaxel, as currently marketed by Sanofi-Aventis, is a white to almost-white powder with an empirical formula of C₄₅H₅₇NO₁₄.C₃H₆O, and a molecular weight of 894.01 (for the acetone solvate)/835.93 (for the solvent free).

As used herein, “injection concentrate” refers to a liquid solution comprising cabazitaxel, or other lipophilic molecule, combined with one or more excipients.

“Single-vial injection concentrate” (sometimes referred to as “sterile liquid in a single vial”) refers to a sterile liquid in a single vial that can be administered by IV to a patient upon dilution with an infusion solution, i.e., no other dilution may be necessary before dilution with the infusion solution.

“Dual-vial injection concentrate” (sometimes referred to as “initial concentrate”) refers to a stable injection concentrate that requires an initial dilution before it can be diluted with an infusion solution for administration to a patient. The initial dilution is performed with a diluent.

“Diluted injection concentrate” (sometimes referred to as “initial diluted solution” or “intermediate concentrate”) refers to the result of the dual-vial injection concentrate combined with a diluent.

“Final dilution for infusion” (sometimes referred to as “final concentrate”) refers to the result of the single-vial injection concentrate or the diluted injection concentrate, combined with an infusion solution. The final dilution for infusion may be ready to be administered to a patient.

“Infusion solution” refers to a sterile isotonic solution, typically stored in a bag or bottle, that is employed to dilute the single-vial injection concentrate or the diluted injection concentrate for administration to a patient.

As used herein, “solubilizer” refers to a solvent that is capable of dissolving cabazitaxel, or a pharmaceutically acceptable salt thereof, or any such lipophilic molecule, to prepare a formulation, such as a single-vial injection concentrate or dual-vial injection concentrate.

As used herein, “hydrotrope” refers to a material that can solubilize cabazitaxel, or a pharmaceutically acceptable salt thereof, or any such lipophilic molecule, if present in a sufficient quantity, and prevents precipitation when the sterile liquid in a single vial or dual-vial injection concentrate is further diluted in an infusion solution. A hydrotrope does not dissolve the drug to the extent as the solubilizer. Two or more hydrotropes can act synergistically on solubility such that the combination can be used as a “solubilizer” in the context of the present invention.

As used herein, “substantially free” refers to the presence of a material in an amount less than about 5% (peak area %), or about 3%, or about 1%, or about 0.5%, or about 0.1%, or about 0% (i.e., totally free) as measured by HPLC with the UV detector set at a specific wavelength.

As used herein, “impurity” refers to any component of a drug product that is not the drug substance or an excipient in the drug product. See ICH Guidelines: Impurities in New Drug Products at 6. An impurity can include any degradant of a drug product.

### Substantially Free of Polysorbates and Polyethoxylated Castor Oil

The formulations of the present invention are substantially free of polysorbates and polyethoxylated castor oil. As a result, the formulations of the present invention should be better tolerated in cancer patients, thereby allowing these patients to take the medication for a longer period of time without dose interruption and/or dose reduction as compared to the current marketed formulation, i.e., JEVTANA. For example, cabazitaxel, formulated without polyethoxylated castor oil or polysorbate 80 may be administered to patients at much higher doses than JEVTANA's dosing range of 25 mg/m² (based on calculation of the Body Surface Area), and/or with less than three weeks between treatments. Moreover, while JEVTANA is currently indicated for prostate cancer in combination with a steroid, a formulation of cabazitaxel without polyethoxylated castor oil or polysorbate 80 may have therapeutic utility to treat other cancers, such as breast, lung, colon, liver, pancreatic, and renal cancer.

Additionally, cabazitaxel formulations substantially free of polysorbates and polyethoxylated castor oil may be administered to patients without pre-medicating with steroids. The reduction or elimination of the steroid pretreatment phase can reduce concerns of immune system depression and other side effects, as well as of interactions with other drugs that the patient may be taking. Also, eliminating polysorbates in the formulation can remove the risk of skin rashes, edema, hypotension and bradycardia.

### Cabazitaxel Formulations

### Injection Concentrates

The present invention relates to formulations of cabazitaxel. Single-vial injection concentrates may comprise cabazitaxel or a pharmaceutically acceptable salt thereof, one or more solubilizers, TPGS, and one or more hydrotropes. Dual-vial injection concentrates may comprise cabazitaxel or a pharmaceutically acceptable salt thereof, and one or more solubilizers. Optionally, the single-vial injection concentrates and the dual-vial injection concentrates may also comprise one or more agents having a pKₐ of about 3 to about 6, one or more antioxidizing agents, and/or water for injection.

Single-vial injection concentrates may comprise cabazitaxel in various amounts according to its use and application, although the most typical amounts are about 8 to about 12 mg/mL, or about 10 mg/mL. Dual-injection concentrates may also comprise cabazitaxel in various amounts, but the most typical are about 40 to about 80 mg/mL, or about 60 mg/mL.

The solubilizers used in the present invention include, but are not limited to, glycofurol, ethanol, acetic acid, and benzyl alcohol. Glycofurol is also known as tetrahydrofurfuryl alcohol polyethylene glycol ether and has the following structure:

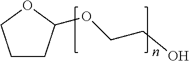

wherein n is on average 2, such as glycofurol 75, but may be other integers such as n=1.

Glycofurol may be combined with a liquid PEG, such as PEG 200, PEG 300, or PEG 400. The resulting mixture may comprise glycofurol and PEG in a ratio % of about 15:85 to about 85:15, or about 30:70 to about 70:30, or about 50:50. Similarly, glycofurol may be combined with ethanol, or ethanol may be combined with PEGs, in the ratios described above. Alternatively, a ternary mixture of glycofurol, ethanol and PEGs at different ratios can be mixed in order to obtain cabazitaxel solutions at desired concentrations.

Ethanol is another solubilizer that can be used in the injection concentrate, as cabazitaxel is soluble in ethanol.

The solubilizer may be present in the either single-vial or dual-vial injection concentrates in an amount sufficient to bring the formulation to a final target volume, i.e., a quantity sufficient (q.s.) amount. For example, if the final target volume of the injection concentrate is about 1 mL, then the solubilizer may q.s. to about 1 mL. Alternatively, one of ordinary skill in the art can determine the appropriate quantity of solubilizer for solubilizing cabazitaxel without undue experimentation.

The one or more agents having a pKₐ of about 3 to about 6 may comprise acid or buffers. The one or more agents having a pKₐ of about 3 to about 6 may be an acid, such as a weak acid, or a buffer. Weak acids for use in the present invention include, but are not limited to citric, acetic, ascorbic, benzoic, lactic, oxalic, propanic, and uric.

The buffer may comprise organic buffer materials that include, without limitation, the following materials together with their conjugate salts (which free compound/salt conjugate may form in situ from either the free compound or the conjugate salt being added alone as known in the art of buffer materials): citric acid, tartaric acid, b-alanine, lactic acid, aspartic acid, g-aminobutyric, succinic acid, oxalic acid, e-aminocaproic acid, acetic acid, propionic acid, and malonic acid.

Hydrotropes of the present invention may include, but are not limited to, PEG such as PEG 200, PEG 300, and PEG 400; propylene glycol (PG); 50% PEG 400/50% PG; LUTROL® (as known as SOLUTOL®) 2% in PEG; tocopherol succinate esters; and acetic acid. In certain embodiments, the hydrotrope may be TPGS, PEG, or a combination thereof.

Antioxidizing agents of the present invention may include, but are not limited to, α-lipoic acid, dihydrolipoic acid, butylated hydroxyanisole (“BHA”), butylated hydroxytoluene (“BHT”), acetylcysteine, ascorbyl palmitate, monothioglycerol, potassium nitrate, sodium ascorbate, sodium formaldehyde sulfoxylate, sodium metabisulfite, sodium bisulfite, vitamin E or a derivative thereof, propyl gallate, edetate (“EDTA”) (e.g., disodium edetate), diethylenetriaminepentaacetic acid (“DTPA”), triglycollamate (“NT”), or a combination thereof. Antioxidants may also comprise thio-amino acids such as methionine, and cysteine.

In some embodiments, the injection concentrates may comprise water for injection. The amount of water for injection may be between about 0.5 and about 10%, or between about 2 and about 8%, of the total volume of the injection concentrates.

The quantity of TPGS, agent having a pKₐ of about 3 to about 6, hydrotrope, and antioxidizing agent may be determined by one of ordinary skill in the art without undue experimentation. For instance, the quantity of TPGS or hydrotrope may be increased if, among other reasons, the final dilution for injection contains cabazitaxel precipitates. As another example, the amount of antioxidizing agents may be increased if, among other reasons, the formulations contain oxidative degradants.

The cabazitaxel injection concentrates can be stored at room temperature (about 15 to about 30° C.) or under refrigerated conditions (about 2 to about 8° C.). The injection concentrates can be stored for at least one-and-a-half to two years at room temperature and longer still under refrigeration.

The cabazitaxel injection concentrate may be lyophilized. For example, cabazitaxel may be dissolved with a hydrotrope (such as TPGS) and a bulking agent (such as soluble saccharide or dissacharide) in a solvent (such as ethanol, dimethyl sulfoxide, tetrahydrofuran, or dioxane), and then the solvent may be removed by lyophilization. The resulting lyophile can be reconstituted in either 13% ethanol or a diluent. The lyophilization procedure can follow methods of lyophilization known in the art.

### Diluted Injection Concentrates

The present invention also relates to diluted injection concentrates, which are the formulations resulting from combining the dual-vial injection concentrates with a diluent.

The diluent may comprise at least one hydrotrope and optionally at least one solubilizer, optionally a tonicity adjuster, and optionally pH-adjusting agent. The hydrotrope and solubilizer may be as described above.

The tonicity adjuster may generally be a solute that alters the ionic concentration of the formulation without having a significant impact on the solubility and stability of cabazitaxel. In various embodiments the tonicity adjuster may be selected from inorganic salts, organic salts, sugars or combinations thereof. Examples of inorganic salts include sodium chloride, potassium chloride, and magnesium chloride. Organic salts may include but are not limited to sodium acetate and sodium citrate. Sugars that are contemplated to be tonicity adjusters within the scope of the present invention may include dextrose, mannitol, sucrose, and the like.

The pH-adjusting agent may be a buffer or an agent having a pKₐ of about 3 to about 6 or an acid. Buffers and agents having a pKₐ of about 3 to about 6 in the diluent are as described above.

Acids may include, but are not limited to, one or more inorganic mineral acids such as hydrochloric, hydrobromic, sulfuric, phosphoric, nitric, and the like; or one or more organic acids such as acetic, succinic, tartaric, ascorbic, citric, glutamic, benzoic, and the like.

In some embodiments, the diluent may comprise water for injection. The amount of water for injection may be between about 1 and about 10%, or between about 2 and 8%, of the total volume of the resulting diluted injection concentrate.

The initial diluted solution may be substantially free of polysorbate 80, CREMOPHOR®, and/or all polyethoxylated vegetable oils (whether totally hydrogenated, partially hydrogenated, or not hydrogenated). In addition, the initial diluted solution may be substantially free of hydroxyalkyl substituted cellulosic polymers.

The diluted injection concentrate may comprise various volumes according to its use. The quantity of TPGS, pH-adjusting agent, hydrotrope, antioxidizing agent, etc., may be determined by one of ordinary skill in the art without undue experimentation, as described above.

The initial diluted solution can be stored at about room temperature for at least about 24 hours, as these conditions will generate no detectable levels of impurities. In addition, after storage at room temperature for up to 24 hours, the initial diluted solution will be clear and colorless. Moreover, the potency of the docetaxel in the initial diluted solution may remain at about 98 to about 100%. The initial diluted solution can also be stored under refrigerated conditions (about 2 to about 8° C.) for even longer periods of time.

### Final Dilution for Infusion

The final dilution for infusion refers to the result of the single-vial injection concentrate or the diluted injection concentrate, combined with an infusion solution. The final dilution for infusion may be ready for administration to a patient.

Infusion solutions used in the final dilution for infusion include, but are not limited to, large volume parenteral such as water for injection, 0.9% sodium chloride solution (or normal saline), and 5% dextrose solution.

The final dilution for infusion may comprise cabazitaxel in an amount of about 0.5 mg/mL to about 0.30 mg/mL, or about 0.10 mg/mL to about 0.26 mg/mL. Therefore, the diluted injection concentrate or the single-vial injection concentrate will be added to an appropriate amount of the infusion solution, which may be dependent on the concentration of cabazitaxel in the diluted injection concentrate or the single-vial injection concentrate. For example, if the diluted injection concentrate or the single-vial injection concentrate contains 8 mg/mL of cabazitaxel, preparation of the final dilution for infusion would involve mixing 1 mL of the diluted injection concentrate or the single-vial injection concentrate with about 79 mL or about 30.8 mL of the infusion solution, to result in a final dilution for infusion having a cabazitaxel concentration of about 0.10 mg/mL or about 0.26 mg/mL, respectively.

The final dilution for infusion can be stored at room temperature for at least about 8 hours, as these conditions generate no detectable levels of impurities. In addition, after storage at room temperature for up to 8 hours, the final dilution for infusion will be clear and colorless. Moreover, the potency of the docetaxel in the final dilution for infusion will remain at 100 or 99%.

### Stability of the Formulations

Stability of the cabazitaxel formulations may be determined by methods known in the art. As example, stability may be assessed by storing the formulations under pre-set conditions for a pre-determined period of time, and then analyzing the formulations after storage for changes in the amount of cabazitaxel or impurity levels.

The changes in the amount of cabazitaxel and impurity levels can be measured using techniques known in the art. For instance, one such technique is high performance liquid chromatography (HPLC). The skilled artisan would understand how to perform HPLC in order to measure the stability of the formulations.

Using HPLC, stability may be assessed by analyzing the generated impurity profile. For example, one measure is the peak area % of the impurities detected by HPLC, or the total peak area % of all impurities detected by HPLC. These measurements may be compared to measurements of the formulation before storage, or may be compared to measurements of a formulation standard (e.g., the formulation for JEVTANA).

### Packages and Kits

The present invention also relates to packages/kits comprising the dual-vial injection concentrate and the diluent of the invention. The dual-vial injection concentrate and the diluent may be housed in separate vessels, such as in vials or containers.

The packages/kits may comprise a withdrawing means such as a syringe, and/or a means to alter the temperature of the vials, e.g., modify the temperature of the vials to room temperature. The packages/kits may also contain instructions for diluting the dual-vial injection concentrate using the diluent.

### Methods of Preparing Cabazitaxel Formulations

The present invention relates to a method of preparing cabazitaxel formulations, including stable cabazitaxel formulations. To prepare a single-vial injection concentrate, the method may comprise combining cabazitaxel, one or more solubilizers, TPGS, one or more agents having a pKₐ of about 3 to about 6, and one or more hydrotropes. To prepare a dual-vial injection concentrate, the method may comprise combining cabazitaxel, one or more solubilizers, and, one or more agents having a pKₐ of about 3 to about 6.

The addition of the components of the single-vial and dual-vial injection concentrates can be achieved by methods known in the art. For example, one or more of the components may be added to each other and then into a common receptacle for mixing, or the components may be added to a common receptacle in a particular order, or the components may be added to a common receptacle simultaneously. In certain embodiments, the cabazitaxel, or other lipophilic molecule, and the solubilizer are combined separately from the other components. In some embodiments, the cabazitaxel, or other lipophilic molecule is dissolved in the solubilizer separately from the other components.

The components of the single-vial and dual-vial injection concentrates may be mixed by methods known in the art. For example, the components can be mixed by simple mixing, or may be mixed with a mixing device continuously, periodically, or a combination thereof. Mixing devices may include, but are not limited to, a magnetic stirrer, shaker, a paddle mixer, homogenizer, and any combination thereof.

The addition and mixing of one or more components of the single-vial and dual-vial injection concentrates may occur under controlled conditions. For example, the addition and mixing of the components may occur under conditions such as under nitrogen or at a particular humidity, etc., or the adding and mixing may occur under certain temperatures. In certain embodiments, the adding and mixing may occur under temperature conditions of about 25° C. to about 80° C. Additionally, the addition and mixing may be under controlled light exposure, such as in yellow light or under protection from direct exposure to light.

After the injection concentrate is prepared, it may be sterilized by methods known in the art. The injection concentrate may undergo aseptic filtration (e.g., using a 0.2 μm disposable pre-sterilized membrane filter).

Additionally, the injection concentrate may be placed into a container (e.g., an intravenous solution bag, bottle, vial, ampoule, or pre-filled sterile syringe). The container may have a sterile access port for piercing by a hypodermic injection needle. In some embodiments, the injection concentrate may be filled in one or more pre-sterilized depyrogeneated vials and stopped aseptically with a pre-sterilized butyl stopper.

The diluted injection concentrate may be formed by mixing the dual-vial injection concentrate and diluent together. In one embodiment the dual-vial injection concentrate may be added to the diluent. In another embodiment, the diluent may be added to the dual-vial injection concentrate. In yet another embodiment, the dual-vial injection concentrate and diluent may be combined together in a pre-sterilized vessel. The dual-vial injection concentrate and diluent may be mixed by repeated inversions, swirling, or other techniques known in the art. Due the absence of polysorbates in the diluent and the dual-vial injection concentrate, little to no foaming occurs during the mixing.

The final dilution for infusion may be prepared by combining a single-vial injection concentrate or a diluted injection concentrate with an infusion solution of the present invention, according to methods known in the art. For example, the single-vial injection concentrate or a diluted injection concentrate may be mixed with an infusion solution in a common receptacle, or the single-vial injection concentrate or the diluted injection concentrate may be injected into an infusion bag containing the infusion solution.

As the present invention is directed to delivery of cabazitaxel, once diluted to appropriate injection (especially infusion, most particularly IV infusion) concentrations, it may be administered in appropriate amounts for treating cabazitaxel responsive conditions known in the art.

Administration of the cabazitaxel final dilution for infusion to the patient may not require premedication with a steroid. Antihistamines are likely not required as a pretreatment but may be administered to the patient as a precaution.

### Methods of Administering Cabazitaxel Formulations

The present invention relates to methods of administering formulations described herein, in particular the final dilutions for infusion. In certain embodiments, the final dilution for infusion is administered by IV as a one-hour infusion at room temperature to patients in need thereof.

In some embodiments, an in-line filter is used during administration. In particular embodiments, the filter is of 0.22 μm nominal pore size.

### Formulations of Other Lipophilic Molecules

The excipients used in formulating cabazitaxel according to the present invention may also be used for formulating other lipophilic molecules. In certain embodiments, the excipients can be used to formulate lipophilic molecules that are typically formulated with polysorbates and polyethoxylated castor oil, as well as phospholipids and cyclodextrins such as 2-hydroxypropyl-beta-cyclodextrin (HPBCD).

Examples of the other lipophilic molecules include, but are not limited to, temsirolimus, amiodarone, sirolimus, cyclosporine, paclitaxel, teniposide, and ixabepilone. Temsirolimus, marketed as TORISEL®, is a kinase inhibitor indicated for the treatment of advanced renal cell carcinoma. TORISEL is provided as an injection concentrate that must be diluted with a diluent before further dilution with an infusion solution for injection. The diluent comprises, among other ingredients, polysorbate 80. In certain embodiments of the invention, temsirolimus may be formulated with one or more solubilizers such as glycofurol and/or benzyl alcohol, TPGS, and one or more hydrotropes such as PEG.

Amiodarone, marketed as CORDARONE®, is indicated for initiation of treatment and prophylaxis of frequently recurring ventricular fibrillation and hemodynamically unstable ventricular tachycardia in patients refractory to other therapy. The IV formulation of CORDARONE contains polysorbate 80. In certain embodiments of the invention, amiodarone may be formulated with TPGS, one or more solubilizers such as glycofurol, and optionally one or more hydrotropes such as PEG.

Sirolimus, marketed as RAPAMUNE®, is an immunosuppressive agent indicated for the prophylaxis of organ rejection in patients 13 years or older receiving renal transplants. The oral solution of RAPAMUNE contains polysorbate 80. In certain embodiments of the invention, sirolimus may be formulated with one or more solubilizers such as glycofurol and/or benzyl alcohol, TPGS, and one or more hydrotropes such as PEG.

Cyclosporine is indicated for the prophylaxis of organ rejection in kidney, liver, and heart allogeneic transplants; for the treatment of patients with severe, active, rheumatoid arthritis where the disease has not adequately responded to methotrexate; and for certain treatments of adult, nonimmunocompromised patients with severe, recalcitrant, plaque psoriasis. The oral solution of cyclosporine contains polyoxyl 40 hydrogenated castor oil. In certain embodiments of the invention, cyclosporine may be formulated with one or more solubilizers such as glycofurol and/or ethanol, TPGS, and optionally one or more hydrotropes such as PEG.

Paclitaxel, marketed as TAXOL®, is indicated as first-line and subsequent therapy for the treatment of advanced carcinoma ovary, certain treatments for breast cancer, and for the second-line treatment of AIDS-related Kaposi's sarcoma. TAXOL is provided as an injection concentrate that must be diluted with an infusion solution for injection. The injection concentrate contains polyoxyethylated castor oil. In certain embodiments of the invention, paclitaxel may be formulated with one or more solubilizers such as glycofurol and/or ethanol, and α-lipoic acid, and optionally TPGS, one or more hydrotropes such as PEG, and one or more agents having a pKₐ of about 3 to about 6 such as citric acid.

Teniposide is marketed as VUMON® and, in combination with other approved anticancer agent, is indicated for induction therapy in patients with refractory childhood acute lymphoblastic leukemia. VUMON is provided as an injection concentrate that must be diluted with an infusion solution for injection. The injection concentrate contains polyoxyethylated castor oil.

Ixabepilone, marketed as IXEMPRA®, is indicated in combination with capecitabine for certain treatments of patients with metastatic or locally advanced breast cancer. IXEMPRA is provided as an injection concentrate that must be diluted with a diluent before further dilution with an infusion solution for injection. The diluent comprises, among other ingredients, polyoxyethylated castor oil. In certain embodiments of the invention, ixabepilone may be formulated with one or more solubilizers such as glycofurol and/or ethanol, and α-lipoic acid, and optionally TPGS, one or more hydrotropes such as PEG, and one or more agents having a pKₐ of about 3 to about 6 such as citric acid.

Everolimus is marketed as CERTICAN®, which is indicated for the prophylaxis of organ rejection in adult patients receiving an allogeneic renal or cardiac transplant, and as AFFINITOR®, which is indicated for the treatment of patients with subependymal giant cell astrocytoma associated with tuberous sclerosis. In certain embodiments of the invention, everolimus may be formulated with one or more solubilizers such as glycofurol and/or benzyl alcohol, TPGS, and one or more hydrotropes such as PEG.

Finally, voriconazole is indicated for the treatment of fungal infections such as invasive aspergillosis, candidemia, esophageal candidiasis, and infections caused by Scedosporium apiospermum. Voriconazole may contain sulfabutylether 2-hydroxypropyl-beta-cyclodextrin. In some embodiments of the invention, voriconazole may be formulated with PEG or PG, one or more agents having a pKₐ of about 3 to about 6 such as citric acid or lactic acid, one or more antioxidants such as thioglycerol, and/or one or more solubilizers such as glycofurol.

These may further comprise one or more antioxidants, one or more buffers and/or pH-adjusting agents and/or agents having a pKₐ of about 3 to about 6, as described for formulating cabazitaxel.

In addition, methods for preparing formulations for lipophilic molecules may include steps described for preparing cabazitaxel formulations.

The invention will now be further described by way of the following non-limiting examples, which further illustrate the invention; such examples are not intended, nor should they be interpreted, to limit the scope of the invention.

### EXAMPLES

### Example 1

Solubility studies are conducted to determine non-toxic solvents that can effectively dissolve cabazitaxel and other lipophilic molecules that are generally formulated with polysorbates and polyethoxylated castor oil, as well as phospholipids and cyclodextrins. Solubility can be assessed using several different solvents that are well-tolerated in subjects. These solvents may include those shown in Table 1.

TABLE 1
Solvents for Testing Solubility
Solvents

TWEEN 80 ®*
PEG 400
Propylene Glycol
50% PEG 400/50% PG
2% Lutrol in PEG 400
TPGS 1000
Ethanol
Benzyl alcohol
Benzyl benzoate
Glycofurol
*polysorbates can be included for comparison

### Example 2

A study is performed to determine the effect of an antioxidant such as α-lipoic acid on the stability of cabazitaxel formulations without polysorbates. These cabazitaxel formulations without polysorbates are shown in Table 2.

TABLE 2
Cabazitaxel formulations without polysorbates.

| Component | Quantity | |
|---|---|---|
| Cabazitaxel | 60 | mg |
| Glycofurol | 1-2 | mL |
| TPGS | 1-2 | g |
| α-lipoic acid | 0-30 | mg |
| Citric acid | 0-30 | mg |
| PEG 400 | q.s. to 6 | mL |

The cabazitaxel formulations are stored at 40° C. over 1 week to 3 months, and/or are stored at 25° C. over 1 week to 3 months. After storage, stability of the formulations is tested using HPLC.

### Example 3

A study to determine the effect of water on the stability of cabazitaxel formulations without polysorbates is conducted. These cabazitaxel formulations are shown in Table 3.

TABLE 3
Cabazitaxel formulations with water.

| Component | Quantity | |
|---|---|---|
| Cabazitaxel | 60 | mg |
| Glycofurol | 1-2 | mL |
| TPGS | 1-2 | g |
| α-lipoic acid | 0-30 | mg |
| Citric acid | 20-30 | mg |
| Water | 0-2 | mL |
| PEG 400 | q.s. to 6 | mL |

The cabazitaxel formulations are stored at 40° C. over 1 week to 3 months, and/or are stored at 25° C. over 1 week to 3 months. After storage, stability of the formulations is tested using HPLC.

### Example 4

A study is conducted to determine the effect on stability of using a different solubilizer, i.e., ethanol, in cabazitaxel formulations without polysorbates. These cabazitaxel formulations are shown in Table 4.

TABLE 4
Cabazitaxel formulations with ethanol as a solubilizer.

| Component | Quantity | |
|---|---|---|
| Cabazitaxel | 60 | mg |
| Ethanol | 1-2 | mL |
| TPGS | 1-2 | g |
| α-lipoic acid | 0-30 | mg |
| Citric acid | 0-30 | mg |
| PEG 400 | q.s. to 6 | mL |

The cabazitaxel formulations are stored at 40° C. over 1, 2, and/or 3 months, and/or are stored at 25° C. over 1, 2, and/or 3 months. After storage, stability of the formulations is tested using HPLC.

### Example 5

Studies to compare the stability of one or more of the cabazitaxel formulations prepared in Examples 3-5 to cabazitaxel formulations with polysorbate 80 are performed. The cabazitaxel formulations with polysorbate 80 are the JEVTANA injection concentrate and diluents. Tables 5 and 6 show the components of the JEVTANA injection concentrate and injection concentrate with diluents, respectively.

TABLE 5
JEVTANA injection concentrate.

| Component | Quantity | |
|---|---|---|
| Cabazitaxel | 60 | mg |
| Polysorbate 80 | 1.5 | mL |

TABLE 6
JEVTANA injection concentrate and diluent.

| Component | Quantity | |
|---|---|---|
| Cabazitaxel | 60 | mg |
| Polysorbate 80 | 1.5 | mL |
| 13% Ethanol | 5.7 | mL |

Stability was compared after storage of the formulations at 40° C. over 1 hour to 3 months, and/or at 25° C. over 1 hour to 3 months. After storage, stability of the formulations is tested using HPLC.

### Example 6

Studies to assess the stability of cabazitaxel formulations without diluted in water for injection are performed. One or more cabazitaxel formulations described in Examples 2-4 are mixed with water for injection to simulate dilution into IV fluids. The diluted cabazitaxel formulations are stored at 40° C. over 1 hour to 1 week, and/or are stored at 25° C. over 1 hour to 1 week. After storage, stability of the formulations is tested using HPLC.

While specific embodiments of the subject invention have been discussed, the above specification is illustrative and not restrictive. One skilled in the art will appreciate that numerous changes and modifications can be made to the invention, and that such changes and modifications can be made without departing from the spirit and scope of the invention.

1. A sterile pharmaceutical formulation for use in treatment of a patient in need thereof, comprising:
(a) cabazitaxel, or a pharmaceutically acceptable salt thereof;
(b) solubilizer, wherein the solubilizer is selected from glycofurol and ethanol;
(c) tocopherol polyethylene glycol succinate (TPGS);
(d) one or more hydrotropes;
(e) optionally one or more agents having a pKₐ of about 3 to about 6; and
(f) optionally one or more antioxidizing agent;
wherein the formulation is substantially free of polysorbates and polyethoxylated castor oil.

2. The pharmaceutical formulation of claim 1, wherein the cabazitaxel is in an amount of about 8 to about 12 mg/mL.

3. (canceled)

4. The pharmaceutical formulation of claim 1, wherein the solubilizer is about 10 to about 35% of the total volume of the formulation.

5. The pharmaceutical formulation of claim 1, wherein the solubilizer is glycofurol.

6. (canceled)

7. The pharmaceutical formulation of claim 1, wherein the one or more hydrotropes is at least polyethylene glycol (PEG).

8-9. (canceled)

10. The pharmaceutical formulation of claim 1, wherein the formulation comprises one or more agents having a pKₐ of about 3 to about 6.

11. (canceled)

12. The pharmaceutical formulation of claim 10, wherein the one or more agents having a pKₐ of about 3 to about 6 is at least an acid.

13. The pharmaceutical formulation of claim 12, wherein the acid is citric acid.

14-20. (canceled)

21. The pharmaceutical formulation of claim 1, wherein the formulation comprises one or more antioxidizing agents.

22. The pharmaceutical formulation of claim 21, wherein the one or more antioxidizing agents is at least α-lipoic acid.

23. (canceled)

24. The pharmaceutical formulation of claim 1, further comprising water for injection.

25. The pharmaceutical formulation of claim 24, wherein the formulation is substantially free of precipitates.

26. A method of preparing a sterile formulation of cabazitaxel, or a pharmaceutically acceptable salt thereof, substantially free of polysorbates and polyethoxylated castor oil, the method comprising combining together:
(a) cabazitaxel, or a pharmaceutically acceptable salt thereof;
(b) solubilizer, wherein the solubilizer is selected from glycofurol and ethanol;
(c) tocopherol polyethylene glycol succinate (TPGS);
(d) one or more hydrotropes;
(e) optionally one or more agents having a pKₐ of about 3 to about 6; and
(f) optionally one or more antioxidizing agents.

27. The method of claim 26, wherein the solubilizer is glycofurol.

28. The method of claim 26, wherein the one or more hydrotropes is at least polyethylene glycol (PEG).

29-34. (canceled)

35. The method of claim 26, wherein the method comprises separately combining the cabazitaxel, or a pharmaceutically acceptable salt thereof, with the solubilizer.

36. (canceled)

37. The method of claim 26, further comprising sterilizing the combination of cabazitaxel, or a pharmaceutically acceptable salt thereof, solubilizer, TPGS, one or more hydrotropes, optionally one or more agents having a pKₐ of about 3 to about 6, and optionally one or more antioxidizing agents.

38. A sterile pharmaceutical formulation for use in treatment of a patient in need thereof, comprising:
(a) cabazitaxel, or a pharmaceutically acceptable salt thereof;
(b) solubilizer, wherein the solubilizer is selected from glycofurol and ethanol;
(c) optionally one or more agents having a pKₐ of about 3 to about 6, wherein the agent(s) maybe acid(s) or buffer(s); and
(d) optionally one or more antioxidizing agents.

39-42. (canceled)

43. The pharmaceutical formulation of claim 38, further comprising a diluent, wherein the diluents comprises ethanol, TPGS, or a combination thereof.

44-48. (canceled)

49. A kit comprising the pharmaceutical formulation of claim 38 and a diluent.

50-53. (canceled)
